(19) United States
(12) Patent Application Publication
(10) Pub. No.: US 2026/0090701 A1
(43) Pub. Date: Apr. 2, 2026

(54) FRICTION REDUCTION IN IMPLANTABLE MEDICAL DEVICES USING FLUIDS AND OILS

(71) Applicant: Boston Scientific Scimed, Inc., Maple Grove, MN (US)

(72) Inventors: Ryan V. Wales, Shrewsbury, MA (US); Scott Edward Corbeil, Litchfield, NH (US); Scott E. Brechbiel, Johnston, RI (US)

(21) Appl. No.: 19/337,181

(22) Filed: Sep. 23, 2025

Related U.S. Application Data
(60) Provisional application No. 63/700,904, filed on Sep. 30, 2024.

(51) Int. Cl. A61B 1/00

(52) U.S. Cl. CPC A61B 1/00135

(57) ABSTRACT

In a general aspect, the techniques described herein relate to a medical device that includes a first elongated member that has an extruded outer jacket having an outer sidewall, where the extruded outer jacket includes a polymer material with a slip agent added to the polymer material.

### CROSS-REFERENCE TO RELATED APPLICATION

This application claims priority to U.S. Provisional Patent Application No. 63/700,904, filed on Sep. 30, 2024, entitled “FRICTION REDUCTION IN IMPLANTABLE MEDICAL DEVICES USING FLUIDS AND OILS”, the disclosure of which is incorporated by reference herein in its entirety.

### TECHNICAL FIELD

This disclosure relates generally to implantable medical devices, including an elongated member and/or a device that receives the elongated member, and, in particular, to the reduction of friction in implantable medical devices using fluids and oils.

### BACKGROUND

A medical device component, such as a guidewire, may move (e.g., slide) relative to another device (e.g., an insertion device, a lumen device, a biliary device, etc.). Frictional forces between the two components may make movement of the elongated device relatively difficult.

### SUMMARY

In a general aspect, the techniques described herein relate to a medical device that includes a first elongated member that has an extruded outer jacket having an outer sidewall, where the extruded outer jacket includes a polymer material with a slip agent added to the polymer material.

Implementations can include one or more of the following features, alone, or in any combination with each other.

For example, the polymer material may not include PFAS.

In another example, the first elongated member can include a metallic wire disposed along an axis of the first elongated member, where the extruded outer jacket is extruded around the metallic wire.

In another example, the medical device can further include: a second elongated member having a lumen configured to receive the first elongated member, where the slip agent reduces a frictional force between the second elongated member and the first elongated member when the first elongated member slides within the lumen of the second elongated member.

In another example, the slip agent can include a silicone oil.

In another example, the slip agent can include a fatty acid.

In another example, the slip agent can include a lecithin.

In another example, the slip agent can include a vegetable oil.

In another example, the slip agent can be mixed into the polymer material before the polymer material is extruded to form the extruded outer jacket.

In another example, the slip agent can migrate from bulk material of the extruded outer jacket to the outer sidewall of the outer jacket.

In another example, the slip agent can migrate to the outer sidewall of the extruded outer jacket after the polymer material is extruded to form the extruded outer jacket.

In another example, the polymer material can have a flexibility that increases by at least 10% as a temperature of the polymer material increases from 20° C. to 40° C.

In another general aspect, the techniques described herein relate to a method that includes applying a lubricant to an outer sidewall of an extruded polymer outer jacket of a first elongated member, where the polymer does not include PFAS, and inserting the first elongated member into a lumen of a second elongated member, where the lubricant reduces a frictional force between the second elongated member and the first elongated member when the first elongated member slides within the lumen of the second elongated member.

Implementations can include one or more of the following features, alone, or in any combination with each other.

For example, the first elongated member can further include a metallic wire disposed along an axis of the first elongated member, and the extruded outer jacket can be extruded around the metallic wire.

In another example, the lubricant can include a silicone oil.

In another example, the lubricant can include a fatty acid.

In another example, the lubricant can include a lecithin.

In another example, the lubricant can include a vegetable oil.

In another example, the lubricant can be mixed into the polymer as a migrating slip agent that migrates from bulk material of the extruded outer jacket to the outer sidewall of the outer jacket before the polymer is extruded to form the extruded outer jacket.

In another example, the polymer can have a flexibility that increases by at least 10% as a temperature of the polymer material increases from 20° C. to 40° C.

### BRIEF DESCRIPTION OF THE DRAWINGS

FIG. 1 is a schematic diagram of a medical device that may be used in surgical procedures.

FIGS. 2A to 2B illustrate examples of elongated members that create a lubricious barrier between two surfaces to reduce frictional forces, which may facilitate movement (e.g., sliding) of the elongated members with respect to a device.

FIGS. 3A to 3C are schematic transparent views of a polymer material impregnated with a slip agent at different points in time during the migration of slip agent molecules from the bulk of a polymer material to a surface of the polymer material.

FIG. 4A is a schematic diagram of a working channel cap that can be used to lubricate an outer sidewall of an elongated member that is inserted into a lumen of another elongated member to reduce frictional forces between the outer sidewall and an inner sidewall that defines the lumen when the elongated member moves within the lumen.

FIG. 4B is a schematic diagram of a lubricant reservoir that can be used to lubricate an outer sidewall of an elongated member that is inserted into a lumen of another elongated member to reduce frictional forces between the outer sidewall and an inner sidewall that defines the lumen when the elongated member moves within the lumen.

FIG. 5 is a flowchart of an example process for reducing friction between an elongated member of a medical device and an insertion tube of an endoscope.

### DETAILED DESCRIPTION

This disclosure relates to medical devices that create a lubricating barrier between two surfaces to reduce frictional forces, which may facilitate movement of an elongated member within a lumen of another elongated member. In some examples, the devices and techniques discussed herein may avoid the use of per- and polyfluoroalkyl (PFAS) substances applied to, or contained within, any components of the medical devices (or one or more of the components of the medical devices). For example, the medical device may include a slip agent (e.g., a non-PFAS material) (e.g., a silicone oil, a fatty acid, a lectin, an oil (e.g., vegetable oil), etc.) that is impregnated in, or applied to, an elongated member (e.g., a guidewire, biasing member, dilator, etc.) or a device (e.g., insertion device, biliary device (e.g., multi-lumen biliary device), scope, etc.), so that when the elongated member is moved through a lumen of another device a lubricating barrier between two sliding surfaces (e.g., the elongated member and the other device) is created, which reduces frictional forces between the sliding surfaces.

The medical device includes an elongated member that includes an outer sidewall. In some example implementations, the elongated member is a guidewire, biasing member, dilator, tube, or a needle, etc. In some example implementations, the outer sidewall of the elongated member is defined by an exterior surface of a polymer outer jacket. The polymer material of the outer jacket can include a slip agent, which can migrate to the outer sidewall to create a lubricating barrier between the elongated member and another device. In another example implementation, a slip agent can be applied to outer sidewall of the elongated member to create the lubricating barrier. In another example implementation, the polymer material of the outer jacket can be formed of a material whose flexibility increases with increasing temperature, so that a distal end of the outer jacket of the elongated member, when inserted into a relatively warm environment, has a greater flexibility than a proximate end of the outer jacket of the elongated member.

The medical device can include a device that defines a lumen that receives the elongated member. The device's lumen may be slightly larger than the size (e.g., diameter) of the elongated member. The elongated member and the device may slide with respect to each other (e.g., the elongated member slides within a lumen of the device). In some examples, the device is an insertion device, scope device, a tubular member, a biliary device, or a multi-lumen device configured to be inserted into a body of a patient, and the elongated member may be inserted into the body via a lumen of the device. Lubricant on an outer sidewall of the elongated member can reduce frictional forces between the elongated member and the lumen as the elongated member moves within the lumen of the device.

In some example implementations, the medical device can include a motion actuator that applies motion to the elongated member to reduce friction between the device and the elongated member. The use of the motion actuator may convert a guidewire into a dynamic guidewire. The motion actuator may be a motor, vibration exciter, a linear actuator, or other mechanized device that may be attached to a guidewire entrance port to allow for motion to be applied to the elongated member while the elongated member is sent through the device. The motion may be rotation motion, vibration (e.g., high frequency vibration), axial movement (e.g., continuous back and forth movement along an axis of the elongated member), and/or a combination of these movements.

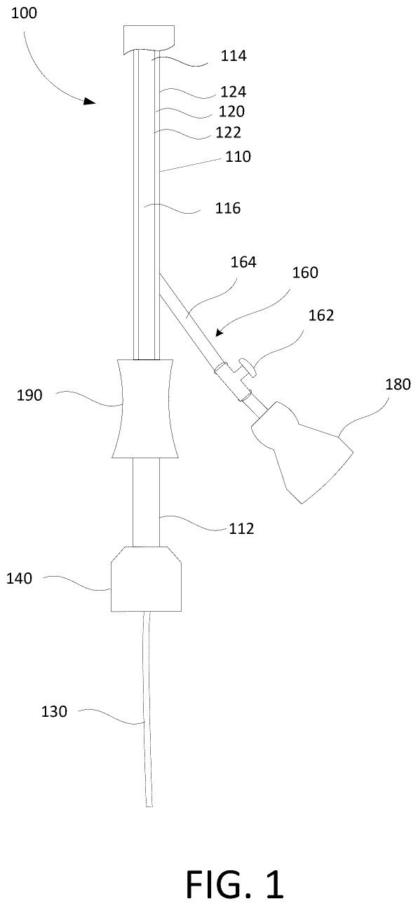

FIG. 1 is a schematic diagram of a medical device 100 that may be used in surgical procedures. For example, in some implementations the medical device 100 may be placed within the body of a patient to create an access, or port, for further medical procedures. More specifically, in some implementations, the medical device 100 may be placed within the body of the patient such that a distal end portion of the medical device 100 is disposed within the body of the patient and a proximal end portion of the medical device 100 extends from the body of the patient. In some implementations, an additional or secondary medical device 130 or a medical instrument may be inserted into the body of the patient via the medical device 100 to perform an additional medical procedure.

In the illustrated implementation, the medical device 100 includes an elongated member 110. The elongated member 110 includes a first or proximal end portion 112 and a second or distal end portion 114. The elongated member defines a lumen 116 that extends from the first or proximal end portion 112 to the second or distal end portion 114. The elongated member 110 includes a sidewall 120 that has an inner surface 122 and an outer surface 124. The inner surface 122 is disposed opposite the outer surface 124, and the inner surface 122 defines the lumen 116. In some implementations, the elongated member 110 is linear or substantially straight or linear. In other implementations, the elongated member 110 is curved or is flexible and may form a curve or have a curved portion. The elongated member 110 or the sidewall 120 may have a non-circular cross-sectional shape. For example, elongated member 110 or the sidewall 120 may have a round or circular cross-sectional shape. In yet other implementations, the elongated member 110 or the sidewall 120 has a different cross-sectional shape, such as square, rectangular, triangular, or any other shape.

The medical device 100 can include a side port 160, and the side port 160 can define a lumen 164. The side port 160 can be coupled to the elongated member 110 or the sidewall 120 such that the lumen 164 of the side port 160 is in fluid communication with the lumen 116 defined by the elongated member 110 or the sidewall 120. The side port 160 is configured to pass fluid into, and receive fluid from, the lumen 116 defined by the elongated member 110 or the sidewall 120. In some implementations, the side port 160 can include a valve 162 that is disposed within the lumen 164 defined by the side port 160. The valve 162 can be configured to help regulate or control the fluid flow through the lumen 164 defined by the side port 160.

The medical device 100 can include a collection member 180 that is coupled to the side port 160 and that is configured to collect material that is disposed within the fluid that flows through or within the lumen 164 of the side port 160. For example, the collection member 180 may be a filter that is configured to collect kidney stones or kidney stone fragments that pass from the body of the patient and through the lumen 164 of the side port 160.

In the illustrated implementation, the medical device 100 includes a handle member or handle portion 190. The handle member or handle portion 190 is coupled to the elongated member 110 or the sidewall 120. The handle member or handle portion 190 is configured to be grasped by a physician or other medical practitioner to place the medical device 100 within the body of a patient or during the use of the medical device 100.

In use, the medical device 100 may be inserted into the body of a patient. In some implementations, the medical device 100 may be inserted into the body of the patient such that the second or distal end portion 114 of the elongated member 110 is disposed within the body of the patient and the first or proximal end portion 112 of the elongated member 110 is disposed outside of the body of the patient (or extends from the body of the patient). For example, in some implementations, the medical device 100 may be placed within the body of the patient such that the second or distal end portion 114 is disposed within a kidney or ureter of the patient. In other implementations, the medical device 100 is placed within the body of the patient such that the second or distal end portion 114 is disposed at a different location within the body of the patient.

The medical device 100 can include an end cap, or a working channel cap, 140 located at the proximal end portion 112 of the elongated member 110. The working channel cap 140 can form a seal at the proximal end portion 112 of the elongated member to maintain a fluid pressure within the lumen of the elongated member and to allow an additional or secondary medical device 130 or a medical instrument to be inserted into the lumen of the elongated member 110.

Once the medical device 100 is disposed within the body of the patient, other medical devices or medical instruments may be inserted into the body of the patient though the lumen defined by the elongated member 110. For example, in some implementations, an elongated member secondary medical device 130 may be inserted into the lumen defined by the elongated member 110 or the sidewall 120 such that the elongated member secondary medical device 130 is disposed within the lumen. Accordingly, the elongated member secondary medical device 130 may be used to perform additional medical procedures once such device 130 is disposed within the body of the patient. In some implementations, the elongated member secondary medical device 130 is a scope. In other implementations, the elongated member secondary medical device 130 is another type of medical device.

The elongated member secondary medical device 130 can have an outer sidewall that is sized and shaped to slide within the lumen of the elongated member 110. A diameter of the outer sidewall of the elongated member secondary medical device 130 can be slightly smaller than the diameter of the lumen. For example, the diameter of the outer sidewall of the elongated member secondary medical device 130 can be less 95% of, less than 90% of, less than 80% of the diameter of the lumen.

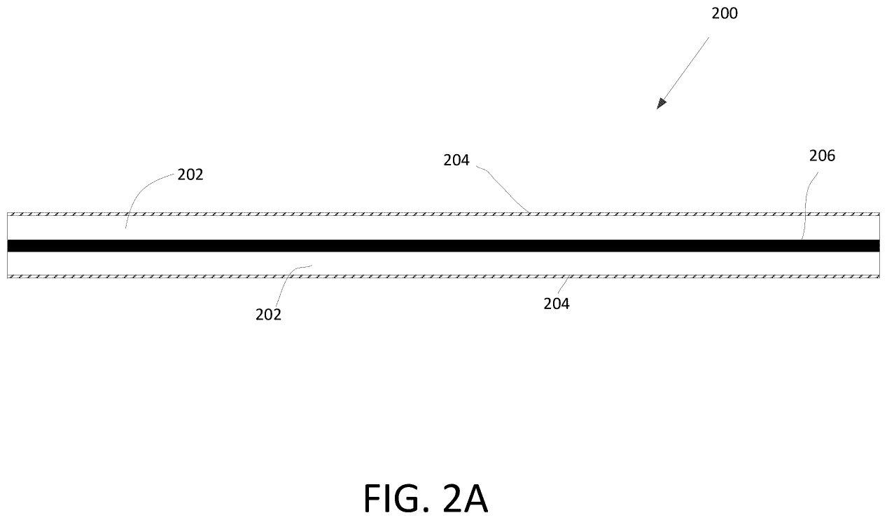

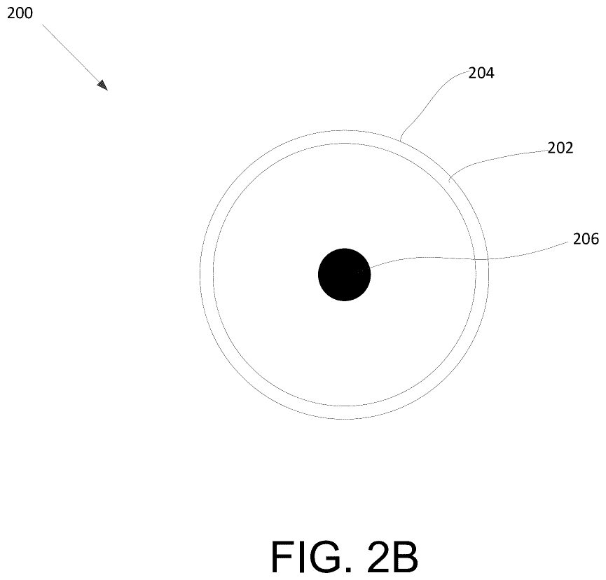

FIG. 2A is a schematic cross-sectional view of an example elongated member 200 that creates a lubricious barrier between two surfaces to reduce frictional forces, which may facilitate movement (e.g., sliding) of the elongated members with respect to a device. The cross-sectional view of FIG. 2A is along an axis of the elongated member. FIG. 2B is a schematic cross-sectional view of the example elongated member 200 with the view being perpendicular to the axis of the elongated member. In some examples, the elongated member 200 may avoid the use of PFAS substances. In some implementations, the elongated member 200 may enable lubricious material to be embedded in the body of an outer jacket 202 of the elongated member 200 and then to migrate to an outer sidewall 204 of the outer jacket, where it creates a lubricating barrier between two sliding surfaces (e.g., the elongated member 200 and an inner sidewall of another elongated member 110, where the inner sidewall of the other elongated member 110 defines a lumen in which the elongated member 200 slides) to reduce frictional forces. In some implementations, the lubricant can be applied directly to the outer sidewall 204 of the elongated member 200 to create a lubricating barrier between two sliding surfaces. In some examples, the material of the elongated member 200 and the lubricant can include any non-PFAS material. In some examples, the lubricant can be a fatty acid, an oil (e.g., vegetable oil, a silicone oil), a lectin, or an amide.

In some implementations, the elongated member 200 can be a guidewire (e.g., a medical guidewire). A guidewire may be a flexible wire used to navigate and access the inside of the body during a minimally invasive medical procedure. In some examples, the elongated member 200 can be a biasing member. In some examples, the elongated member 200 can be a dilator.

In some implementations, the elongated member 200 can include a metallic wire 206, which can run along a central axis of the elongated member. In some examples, the metallic wire 206 includes a Nitinol material. The elongated member 200 can include an outer jacket 202 that includes a polymer material and that surrounds the metallic wire 206. The outer jacket 202 has an outer sidewall 204 that defines an outer diameter or maximum cross-section of the elongated member. The outer jacket 202 can include non-PFAS material. For example, the outer jacket 202 can include Polyphtalamide, Polyetheretherketone, Polyphenylensulfide, Polyamide, Polyolefin, Polyurethane, or other non-PFAS materials.

The outer jacket 202 of the elongated member 200 can be extruded over the metallic wire 206 in an extrusion process. In some example implementations, polymer material can be heated to a temperature above a glass temperature (T_{g}) of the polymer material, so that the polymer material can flow and be formed into the shape of the outer jacket 202. A slip agent can be included in the polymer material that is used in the extrusion process, where the slip agent, at least in part, can form the lubricious barrier on the outer sidewall 204 of the outer jacket 202. The slip agent can include a material that migrates from the bulk material of the outer jacket 202 to the outer sidewall 204.

A slip agent compound can have a part that is soluble in the organic polymer material of the outer jacket 202 and a part that is insoluble. During the molten phase of the extrusion process of the outer jacket 202, the slip agent is soluble in the molten polymer material and homogeneously dispersed in the polymer material. Upon crystallization of the polymer material, after the polymer material has cooled, the solubility limit of the slip agent is exceeded and the slip agent migrates from the matrix of the polymer material to the surface of the outer sidewall 204 of the outer jacket 202. After migration of the slip agent, which may take days or weeks, the concentration of the slip agent within the structure of the outer jacket 202 reaches an equilibrium and a continuous coating of at least one layer of slip agent molecules is formed at, or on, the surface of the outer sidewall 204.

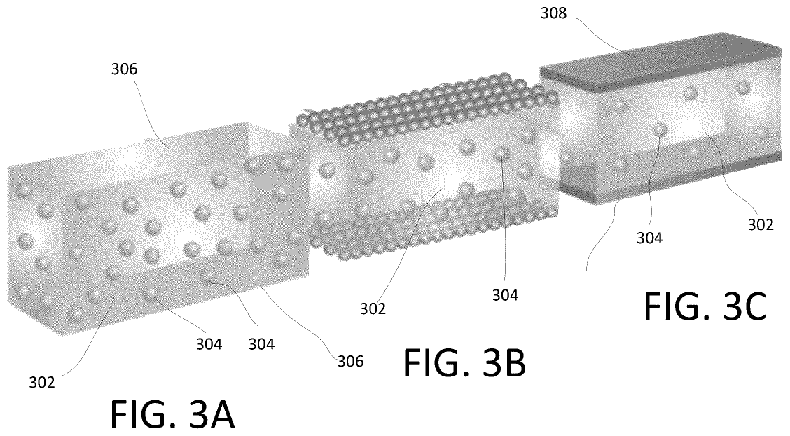

FIGS. 3A, 3B, 3C are schematic transparent views of a polymer material 302 impregnated with a slip agent at different points in time during the migration of slip agent molecules 304 from the bulk of a polymer material 302 to a surface of the polymer material. FIG. 3A corresponds to a time at which polymer material 302 in a molten state and/or has a temperature above a glass temperature of the material and the slip agent molecules 304 are homogeneously dispersed in the polymer material 302. FIG. 3B corresponds to a time at which polymer material 302 has cooled from its molten state and/or has a temperature below the glass temperature of the material. In such a state, a significant proportion of the slip agent molecules 304 have migrated from the bulk of the polymer material 302 to surfaces 306 of the polymer material. FIG. 3C corresponds to a time at which polymer material 302 has cooled additionally from its molten state and sometime after the time the polymer agent has reached the state shown in FIG. 3B. As shown in FIG. 3C, an even larger proportion of the slip agent molecules 304 have migrated from the bulk of the polymer material 302 to the surfaces 306 of the polymer material than the proportion shown in FIG. 3B. As shown in FIG. 3C, the slip agent molecules 304 that have migrated from the bulk of the polymer material 302 to the surfaces 306 of the polymer material for lubricant layers 308 on the surfaces of the polymer material.

In general, slip agents mixed with the polymer material 302 and that facilitate migration or blooming to the polymer surface of the polymer material include low molecular weight compounds that have a low compatibility with the primary polymer. This allows the materials to discretely mix within the polymer matrix and subsequently to bloom to the surface. In some implementations, the slip agent can include a polyethylene wax. In some implementations, the slip agent can include fatty acid amides, including primary and/or secondary amides. For example, primary, unsaturated fatty acid amides such as erucamide and oleamide have relatively high migration rates and can be used as slip agents. In another example, secondary amides, for example, oleyl palmitamide, are less volatile and have relatively slow migration rates, but also can be used as the slip agent, because the elongated member is likely to be used sufficiently long after the slip agent has migrated to the surface of the outer sidewall 204 of the outer jacket 202.

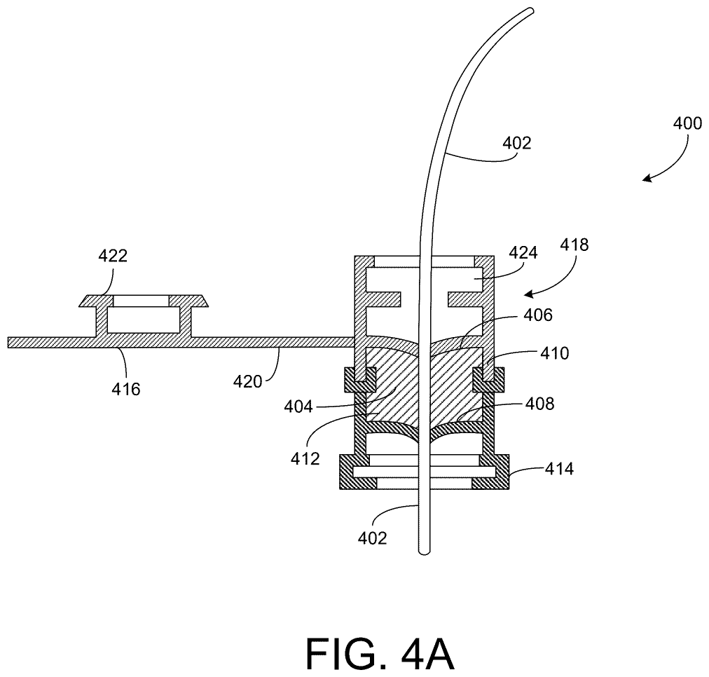

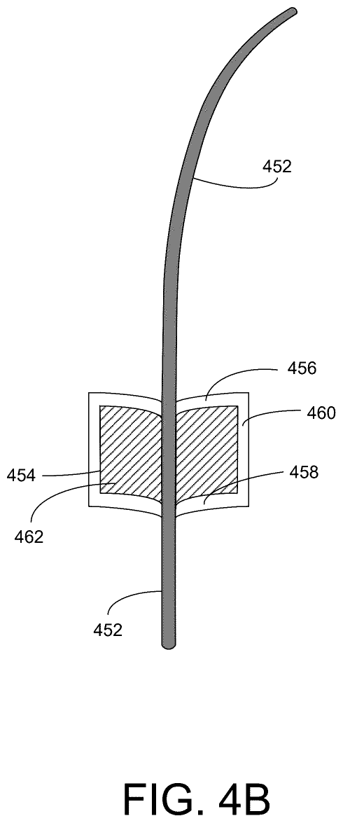

In another implementation, the lubricant can be applied directly to the outer sidewall 204 of the elongated member 200 to create a lubricating surface to facilitate movement of the elongated member 200 along, or within, another structure (e.g., within a lumen of another elongated member). The lubricant can be applied to the outer sidewall 204 of the elongated member in a variety of ways. For example, the lubricant can be applied to the outer sidewall 204 by submerging the elongated member in the lubricant, or by wiping the lubricant onto the outer surface. In another example, as explained in more detail in connection with FIGS. 4A and 4B, the lubricant can be applied to the outer surface at the time the elongated member is inserted into the lumen of another elongated member.

In some implementations, the lubricant applied to the surface of the outer sidewall 204 can include a fatty acid, a lecithin, or another biologically derived lubricants as surface coatings. In some particular examples, the lubricant can include a fatty acid rich flax seed oil, olive oil, or soy-derived lecithin. In some cases, the lubricant, for example, such as, a coco oil, can have a solidification temperature below 25° C., such that it can be applied to the elongated member 200 in a solid form and then can be liquified when, or just before, it is used (for example, with the body of a person).

FIG. 4A is a schematic diagram of a working channel cap 400 that can be used to lubricate an outer sidewall of an elongated member 402 that is inserted into a lumen of another elongated member to reduce frictional forces between the outer sidewall and an inner sidewall that defines the lumen when the elongated member 402 moves within the lumen. The working channel cap 400 is an example of a possible implementation of the working channel cap 140 of FIG. 1.

The working channel cap 400 can include a reservoir 404 defined by a first diaphragm 406, a second diaphragm 408, and one or more sidewalls (e.g., a cylindrical sidewall). The first diaphragm 406 and the second diaphragm 408 can made of a flexible material (e.g., silicone, rubber, or the like), and each diaphragm can include a normally closed valve or orifice through which the elongated member 402 can be passed as the elongated member is inserted into a lumen of another elongated member, for example, the lumen 116 of the elongated member 110 of FIG. 1. The reservoir can contain a lubricant 412. In some implementations, the lubricant 412 can include a fatty acid, an oil (e.g., vegetable oil), or any type of fluid.

The working channel cap 400 can include a flange 414 that can couple the working channel cap 400 to a proximate end of an elongated member, such as, for example, the proximate end 112 of the elongated member 110 of FIG. 1. The working channel cap 400 can include a lid 416 that can be connected to a body 418 of the working channel cap 400 by a tether 420. The tether can include a flexible material, such as, for example, an elastomeric or polymer material that can be manipulated to couple a flange 422 of the lid 416 to an undercut feature 424 of the body 418 of the working channel cap 400 to close off access to the reservoir 404.

With the lid 416 removed from the body 418 of the working channel cap 400 and the reservoir 404 accessible, and with the working channel cap 400 coupled to another elongated member that contains a lumen, the elongated member 402 can be moved through the lubricant-containing reservoir 404 as the elongated member 402 is inserted into the lumen. As the elongated member 402 passes though the lubricant-containing reservoir 404, an outer sidewall of the elongated member 402 can be coated with lubricant 412 from the reservoir 404, and the lubricant 412 can serve to reduce frictional forces between the outer sidewall of the elongated member 402 and an inner sidewall that defines the lumen when the elongated member 402 moves within the lumen.

In some implementations, the second diaphragm 408 can include an elastomeric material incorporating one or more slits that allow both expansion of the second diaphragm 408 around the elongated member 402 as it is passed through the second diaphragm 408, and that also can separate from each other to create small gaps that allow small amounts of lubricant 412 to pass through the second diaphragm 408 on the outer sidewall 410 of the elongated member 402. The slits may further be combined with a self-scaling orifice to facilitate passing of the elongated member 402 and transfer of the lubricant 412 to the elongated member 402.

In some implementations, lubricant reservoir can be provided, which is not part of a working channel cap, and an elongated member can be passed through the lubricant reservoir before it is inserted into a lumen. For example, FIG. 4B is a schematic diagram of a lubricant reservoir 454 that can be used to lubricate an outer sidewall of an elongated member that is inserted into a lumen of another elongated member to reduce frictional forces between the outer sidewall and an inner sidewall that defines the lumen when the elongated member moves within the lumen.

The lubricant reservoir 454 can be defined by a first diaphragm 456, a second diaphragm 458, and one or more sidewalls 460 (e.g., a cylindrical sidewall). The first diaphragm 456 and the second diaphragm 458 can be made of a flexible material (e.g., silicone, rubber, or the like), and each diaphragm can include a normally closed valve or orifice through which an elongated member 452 can be passed as the elongated member is inserted into a lumen of another elongated member, for example, the lumen 116 of the elongated member 110 of FIG. 1. The reservoir can contain a lubricant 462. In some implementations, the lubricant 462 can include a fatty acid, an oil (e.g., vegetable oil), or any type of fluid.

Before the elongated member 452 is inserted into another elongated member that contains a lumen, the elongated member 452 can be moved through the lubricant-containing reservoir 454. As the elongated member 452 passes though the lubricant-containing reservoir 454, an outer sidewall of the elongated member 452 can be coated with lubricant 462 from the reservoir 454, and the lubricant 462 can serve to reduce frictional forces between the outer sidewall of the elongated member 452 and an inner sidewall that defines the lumen when the elongated member 452 moves within the lumen. In some implementations, the reservoir 454 can be placed adjacent to a working channel of an endoscope and the elongated member 452 can be threaded through the lubricant-containing reservoir before it is fed into a lumen in the working channel of the endoscope.

In some implementations, the second diaphragm 458 can include an elastomeric material incorporating one or more slits that allow both expansion of the second diaphragm 458 around the elongated member 452 as it is passed through the second diaphragm 458, and that also can separate from each other to create small gaps that allow small amounts of lubricant 462 to pass through the second diaphragm 458 on the outer sidewall of the elongated member 452. The slits may further be combined with a self-sealing orifice to facilitate passing of the elongated member 452 and transfer of the lubricant 462 to the elongated member 452.

Referring again to FIGS. 2A and 2B, in some implementations, the polymer material of the outer jacket 202 can have a flexibility that varies with temperature, so that a distal end of the outer jacket, which is inserted into a body of a patient, becomes more flexible than a proximate end of the outer jacket, which remains outside of the body. For example, when the elongated member 200 is fed through a lumen of another elongated member (for example, the lumen 116 of the elongated member 110 of FIG. 1), heat from the body can increase the temperature of the polymer material of the outer jacket 202, and this increase in temperature can cause the outer jacket to become more flexible the more distal it is to the biopsy channel. The proximal section of the elongated member that remains outside of the endoscope in contact with room temperature air can be relatively rigid because of its lower temperature. Because of this, the pushability of the elongated member 200 in the lumen may be improved.

In some implementations, to provide such a temperature-dependent flexibility, the outer jacket 202 can include thermopolymers, such as flexible poly vinyl chloride, In another example, the outer jacket 202 can include a polymer that is amorphous in nature, such as for example, PVAC or another polymer in the polyvinyl ester family. In some implementations, the polymer material of the outer jacket can have a flexibility that increases by at least 10% as a temperature of the polymer material increases from 20° C. to 40° C.

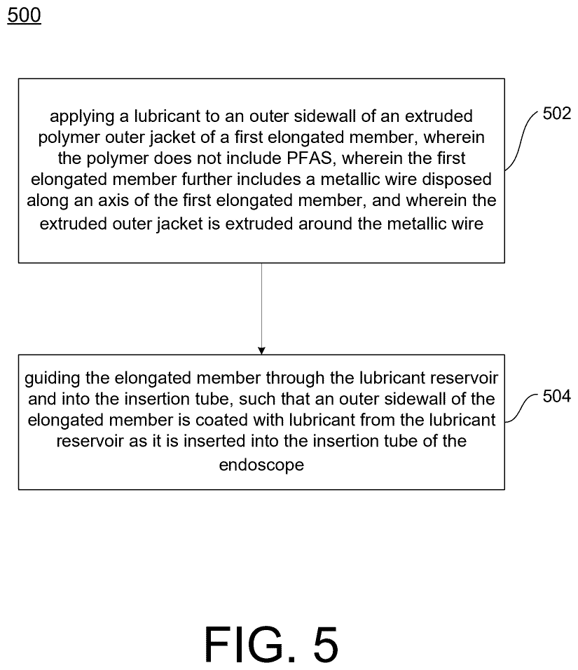

FIG. 5 is a flowchart of an example process 500 for reducing friction between an elongated member of a medical device and an insertion tube of an endoscope. The process 500 includes applying a lubricant to an outer sidewall of an extruded polymer outer jacket of a first elongated member (502), where the polymer does not include PFAS, and where the first elongated member includes a metallic wire disposed along an axis of the first elongated member, and where the extruded outer jacket is extruded around the metallic wire. The process 500 also includes inserting the first elongated member into a lumen of a second elongated member (504), where the lubricant reduces a frictional force between the second elongated member and the first elongated member when the first elongated member slides within the lumen of the second elongated member.

Detailed implementations are disclosed herein. However, it is understood that the disclosed implementations are merely examples, which may be embodied in various forms. Therefore, specific structural and functional details disclosed herein are not to be interpreted as limiting, but merely as a basis for the claims and as a representative basis for teaching one skilled in the art to variously employ the implementations in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the implementations are directed to bodily implants. The term patient or user may hereafter be used for a person who benefits from the medical device or the methods disclosed in the present disclosure. For example, the patient can be a person whose body is implanted with the medical device or the method disclosed for operating the medical device by the present disclosure. For example, in some implementations, the patient may be a human.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the implementations.

What is claimed is:

1. A medical device comprising:
a first elongated member, the first elongated member including an outer jacket having an outer sidewall,
wherein the outer jacket includes a polymer material with a slip agent added to the polymer material.

2. The medical device of claim 1, wherein the polymer material does not include PFAS.

3. The medical device of claim 1,
wherein the first elongated member further includes a metallic wire disposed along an axis of the first elongated member, and
wherein the outer jacket is extruded around the metallic wire.

4. The medical device of claim 1, further comprising:
a second elongated member having a lumen configured to receive the first elongated member, the slip agent reducing a frictional force between the second elongated member and the first elongated member when the first elongated member slides within the lumen of the second elongated member.

5. The medical device of claim 1, wherein the slip agent includes a silicone oil.

6. The medical device of claim 1, wherein the slip agent includes a fatty acid.

7. The medical device of claim 1, wherein the slip agent includes a lecithin.

8. The medical device of claim 1, wherein the slip agent includes a vegetable oil.

9. The medical device of claim 1, wherein the slip agent is mixed into the polymer material before the polymer material is extruded to form the outer jacket.

10. The medical device of claim 9, wherein the slip agent migrates from bulk material of the outer jacket to the outer sidewall of the outer jacket.

11. The medical device of claim 1, wherein the slip agent migrates to the outer sidewall of the outer jacket after the polymer material is extruded to form the outer jacket.

12. The medical device of claim 1, wherein the polymer material has a flexibility that increases by at least 10% as a temperature of the polymer material increases from 20° C. to 40° C.

13. A method comprising:
applying a lubricant to an outer sidewall of a polymer outer jacket of a first elongated member, wherein the polymer does not include PFAS; and
inserting the first elongated member into a lumen of a second elongated member, wherein the lubricant reduces a frictional force between the second elongated member and the first elongated member when the first elongated member slides within the lumen of the second elongated member.

14. The method of claim 13,
wherein the first elongated member further includes a metallic wire disposed along an axis of the first elongated member, and
wherein the outer jacket is extruded around the metallic wire.

15. The method of claim 13, wherein the lubricant includes a silicone oil.

16. The method of claim 13, wherein the lubricant includes a fatty acid.

17. The method of claim 13, wherein the lubricant includes a lecithin.

18. The method of claim 13, wherein the lubricant includes a vegetable oil.

19. The method of claim 13, wherein the lubricant is mixed into the polymer as a migrating slip agent that migrates from bulk material of the outer jacket to the outer sidewall of the outer jacket before the polymer is extruded to form the outer jacket.

20. The method of claim 13, wherein the polymer has a flexibility that increases by at least 10% as a temperature of the polymer increases from 20° C. to 40° C.
